(19) United States
(12) Patent Application Publication
(10) Pub. No.: US 2019/0220738 A1
(43) Pub. Date: Jul. 18, 2019

(54) SKIN ANALYSIS SYSTEM AND METHOD

(71) Applicant: Amit Flank, Beachwood, OH (US)

(72) Inventor: Amit Flank, Beachwood, OH (US)

(21) Appl. No.: 15/874,203

(22) Filed: Jan. 18, 2018

(51) Int. Cl. G06N 3/08; A61B 5/00; G06T 7/00

(52) U.S. Cl. CPC G06N 3/08; A61B 5/444; G16H 30/40; G06T 2207/30088; G06T 7/0012

(57) ABSTRACT

Disclosed embodiments improve the technical field of machine learning and further provide a skin condition analysis system. A user, using a client device such as a smartphone, takes one or more images of a skin condition. The images are uploaded to a skin condition analysis server that implements an evaluation neural network that is trained to analyze skin conditions. The evaluation neural network has hyperparameters that are adjusted utilizing a second neural network to achieve a higher level of diagnostic effectiveness. The client device then retrieves the diagnosis, and renders the diagnosis, along with a corresponding treatment regiment, on a user interface of the client device. In this way, access to effective skin condition treatment is greatly increased. This can serve to provide quicker remedies, thereby reducing overall healthcare costs.

### FIELD OF THE INVENTION

The present invention relates generally to machine learning, and more particularly, to a skin analysis system and method based on machine learning.

### BACKGROUND

Skin health is important not only for one's appearance, but even more importantly because skin performs many essential tasks for the human body. Skin protects the body from the many viruses and bacteria that people are constantly exposed to. It also protects people from the ultraviolet light from the sun, which can damage cells. Healthy skin produces vitamin D when exposed to the sun, and vitamin D is important for many body functions. Having healthy skin also helps the body keep its temperature at a constant level. Healthy skin also helps people react better to important changes in their surroundings by enable them to feel pain or pressure. Thus, healthy skin is an important component of overall well-being.

The outermost skin layer, referred to as the epidermis, is a first line of defense against intruders, such as germs, and the elements. The epidermis protects the second layer of skin, the dermis, which contains important structures like sweat glands and hair follicles.

A variety of skin conditions exist that can affect the health of the skin. These include, but are not limited to, rashes, itchy skin, skin fungus or infection, skin bumps or skin tags, melanomas, and/or other skin cancers. Effective diagnosis and treatment of these skin conditions can be vital in restoring the health of the skin, and overall well-being of a patient.

### SUMMARY

In one embodiment, there is provided a computer-implemented method for training a neural network, comprising: obtaining a plurality of training images; providing the plurality of training images to an evaluation neural network, wherein the evaluation neural network comprises an input layer, an output layer, and one or more hidden layers configured between the input layer and the output layer; providing an output of the evaluation neural network into a loss function analyzer, wherein the loss function analyzer is configured and disposed to evaluate a loss function of the evaluation neural network; providing an output of the loss function analyzer to an optimizing preprocessor, wherein the optimizing preprocessor is configured and disposed to adjust weights based on the loss function analyzer input; providing an output of the optimizing preprocessor to a second neural network, wherein the second neural network is configured and disposed to compute partial derivatives of one or more hyperparameters of the evaluation neural network; providing an output of the second neural network to a parameter adjustment module, wherein the parameter adjustment module is configured and disposed to provide one or more updated hyperparameter values to the evaluation neural network based on the partial derivatives and a previous set of hyperparameter values; applying the one or more updated hyperparameters to the evaluation neural network; and training the evaluation neural network, using the plurality of training images.

In another embodiment, there is provided electronic computation device, comprising: a processor, a memory coupled to the processor, the memory containing instructions, that when executed by the processor, perform the steps of: obtaining a plurality of training images; providing the plurality of training images to an evaluation neural network, wherein the evaluation neural network comprises an input layer, an output layer, and one or more hidden layers configured between the input layer and the output layer; providing an output of the evaluation neural network into a loss function analyzer, wherein the loss function analyzer is configured and disposed to evaluate a loss function of the evaluation neural network; providing an output of the loss function analyzer to an optimizing preprocessor, wherein the optimizing preprocessor is configured and disposed to adjust weights based on the loss function analyzer input; providing an output of the optimizing preprocessor to a second neural network, wherein the second neural network is configured and disposed to compute partial derivatives of one or more hyperparameters of the evaluation neural network; providing an output of the second neural network to a parameter adjustment module, wherein the parameter adjustment module is configured and disposed to provide one or more updated hyperparameter values to the evaluation neural network based on the partial derivatives and a previous set of hyperparameter values; applying the one or more updated hyperparameters to the evaluation neural network; and training the evaluation neural network, using the plurality of training images.

In yet another embodiment, there is provided a computer program product for an electronic computation device comprising a computer readable storage medium having program instructions embodied therewith, the program instructions executable by a processor to cause the electronic computation device to perform the steps of: obtaining a plurality of training images; providing the plurality of training images to an evaluation neural network, wherein the evaluation neural network comprises an input layer, an output layer, and one or more hidden layers configured between the input layer and the output layer, providing an output of the evaluation neural network into a loss function analyzer, wherein the loss function analyzer is configured and disposed to evaluate a loss function of the evaluation neural network; providing an output of the loss function analyzer to an optimizing preprocessor, wherein the optimizing preprocessor is configured and disposed to adjust weights based on the loss function analyzer input; providing an output of the optimizing preprocessor to a second neural network, wherein the second neural network is configured and disposed to compute partial derivatives of one or more hyperparameters of the evaluation neural network; providing an output of the second neural network to a parameter adjustment module, wherein the parameter adjustment module is configured and disposed to provide one or more updated hyperparameter values to the evaluation neural network based on the partial derivatives and a previous set of hyperparameter values; applying the one or more updated hyperparameters to the evaluation neural network; and training the evaluation neural network, using the plurality of training images.

### BRIEF DESCRIPTION OF THE DRAWINGS

The structure, operation, and advantages of the present invention will become further apparent upon consideration of the following description taken in conjunction with the accompanying figures (FIGs.). The figures are intended to be illustrative, not limiting.

Certain elements in some of the figures may be omitted, or illustrated not-to-scale, for illustrative clarity. The cross-sectional views may be in the form of“slices”, or “near-sighted” cross-sectional views, omitting certain background lines which would otherwise be visible in a “true” cross-sectional view, for illustrative clarity. Furthermore, for clarity, some reference numbers may be omitted in certain drawings.

FIG. 1 is a block diagram of a system in accordance with embodiments of the present invention.

FIG. 2 is a block diagram of a client device in accordance with embodiments of the present invention.

FIG. 3 is a flowchart indicating process steps for embodiments of the present invention.

FIG. 4 is a flowchart indicating additional process steps for embodiments of the present invention.

FIG. 5 is a block diagram of an evaluation neural network in accordance with embodiments of the present invention.

FIG. 6A is a block diagram of a feedback network in accordance with embodiments of the present invention.

FIG. 6B is a flow diagram of a feedback network in accordance with additional embodiments of the present invention.

FIG. 7 is an exemplary user interface showing results in accordance with embodiments of the present invention.

FIG. 8A-FIG. 8F show examples of training images in accordance with embodiments of the present invention.

### DETAILED DESCRIPTION

Disclosed embodiments provide a system and method for skin condition diagnosis. An evaluation neural network is trained using multiple training images over multiple iterations. At various iterations, a second neural network is used to adjust the hyperparameters of the evaluation neural network, providing the potential for a more effectively trained machine learning skin condition analysis system.

Current dermatological treatment includes assessing a series of features (often referred to as symptoms) and the various intensities. As an example, for acne, an intensity of the feature “pimples” could include the number of pimples. Diagnosis includes mapping a large range of features and their corresponding intensity values to some disease classification (e.g. acne). The treatment protocol includes mapping that disease to some treatment. In this way, a wide range of various features and their corresponding intensities are mapped to a single disease classification (e.g. regardless of how many pimples a patient has, he is prescribed the same acne medication at the same dosage). This results in a “one size fits all” treatment where treatment includes both the prescribed action and the corresponding dosage of that action, be it physical therapy, some medication, etc. The “one size fits all” treatment may not be optimally effective on some patients. At best, there may be some small sub-group of diseases within some disease category (e.g. various versions of acne) and some limited personalization based on large heterogeneous groups (e.g. different dosages for men and women) that map to different treatments, but even then, there is still a very large information loss during that mapping. This is partly due to human limitation (a doctor is unlikely to be able to distinguish between 50 and 60 pimples or small differences in the color of those pimples) that necessitate such generalization.

However, neural networks provide a mechanism for overcoming this limitation. Thus, with disclosed embodiments, instead of mapping some set of features to a disease and then mapping that disease to some treatment, disclosed embodiments bypass the initial mapping, and instead map some set of features and their corresponding intensities directly to a treatment. Disclosed embodiments accomplish this by training a neural network directly on the results of a treatment. That is, the neural network is fed some set of features and their corresponding intensities before some treatment and the neural network is trained using the results of those treatments with some desirability value via supervised learning. The neural network is then used to take in some novel set of features and their corresponding intensities and have it directly output some treatment recommendation. This enables disclosed embodiments to derive some personalized treatment option for any unique set of features and their corresponding intensities. Moreover, disclosed embodiments, through continued training, can continuously improve the performance and personalization of this network by feeding it additional information (e.g. medical history of the patient, preexisting conditions, etc.) since the network can simply interpret that additional information as another set of features with some intensity values. Thus, disclosed embodiments can enable a new level of dermatological diagnosis and treatments.

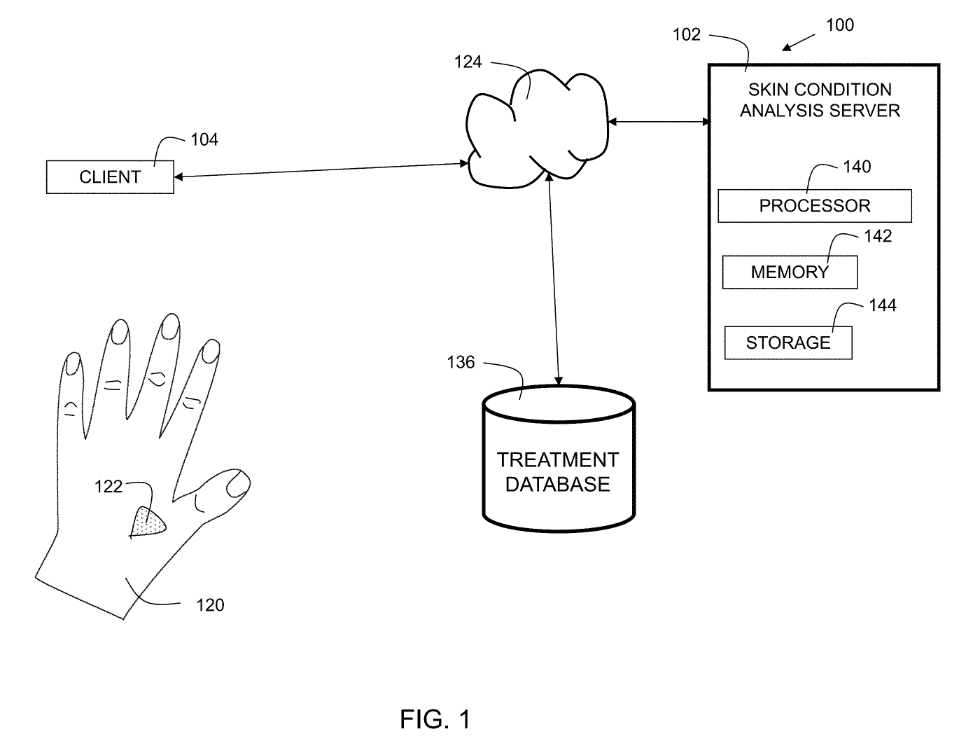

FIG. 1 is a block diagram of a system in accordance with embodiments of the present invention. System 100 includes a skin condition analysis server 102. Skin condition analysis server 102 is an electronic computation device. In embodiments, the skin condition analysis server 102 is implemented as a computer comprising a processor 140, and memory 142 coupled to the processor. The memory 142 may be a non-transitory computer readable medium. Memory 142 may include RAM, ROM, flash, EEPROM, or other suitable storage technology. The memory 142 contains instructions, that when executed by processor 140, enable communication with a variety of other devices and data stores. In embodiments, network 124 may include the Internet.

Storage 144 may include one or more magnetic hard disk drives (HDD), solid state disk drives (SSD), optical storage devices, tape drives, and/or other suitable storage devices. In embodiments, storage 144 may include multiple hard disk drives configured in a RAID (redundant array of independent disks) configuration. In embodiments, the RAID configuration can include a RAID 1 configuration in which data is copied seamlessly and simultaneously, from one disk to another, creating a replica, or mirror. If one hard disk drive becomes inoperable, another hard disk drive continues to operate, providing a level of fault tolerance.

In other embodiments, the RAID configuration can include a RAID 5 configuration in which data and parity are striped across three or more disks. If one hard disk drive within the array gets an error or starts to fail, data is recreated from this distributed data and parity block, seamlessly and automatically. This allows disclosed embodiments to remain operational even when one hard disk drive fails.

In yet other embodiments, the RAID configuration can include a RAID 6 configuration. This configuration is similar to the RAID 5 configuration, with the added enhancement of utilizing more parity blocks than RAID 5, allowing for more hard disk drives to fail while still remaining operational.

In yet other embodiments, the RAID configuration can include a RAID 10 configuration. RAID 10 is a combination of RAID 1 and 0 and is often denoted as RAID 1+0. It combines the mirroring of RAID 1 with the striping of RAID 0, thereby achieving a higher level of performance. Other redundancy schemes are possible with disclosed embodiments.

In yet other embodiments, the skin condition analysis server 102 may be implemented as a virtual machine (VM). In some embodiments, the virtual machine may be hosted in a cloud computing environment.

A client device 104 is also connected to network 124. In embodiments, client device 104 may include, but is not limited to, a desktop computer, a laptop computer, a tablet computer, a mobile phone (e.g. smartphone), and/or other suitable electronic computing device. Note that while one client device 104 is shown in FIG. 1, in practice, multiple client devices may concurrently establish connections with skin condition analysis server 102 in accordance with embodiments of the present invention.

The term “Internet” as used herein refers to a network of networks which uses certain protocols, such as the TCP/IP protocol, and possibly other protocols such as the hypertext transfer protocol (HTTP) for hypertext markup language (HTML) documents that make up the World Wide Web (web). The physical connections of the Internet and the protocols and communication procedures of the Internet are well known to those of skill in the art. Access to the Internet can be provided by Internet service providers (ISP). Users on client systems, such as client device 104 obtains access to the Internet through the Internet service providers. Access to the Internet allows users of the client computer systems to exchange information, receive and send e-mails, and view documents, such as documents which have been prepared in the HTML format. These documents are often provided by web servers which are considered to be “on” the Internet. Often these web servers are provided by the ISPs, although a computer system can be set up and connected to the Internet without that system being also an ISP as is well known in the art.

System 100 may further include a treatment database 136. The treatment database 136 may comprise multiple records, where each record includes one or more possible treatments for a given skin condition. The treatment database 136 may be implemented as a relational database, utilizing a Structured Query Language (SQL) format, or another suitable database format. In some embodiments, the treatment database 136 may include multiple entries for a particular condition, based on various factors such as severity, age of patient, gender of patient, size of patient, and/or preexisting medical conditions. In this way, a more individualized treatment plan can be achieved.

In use, the client device 104 is used to acquire an image. As an example, a human hand 120 is shown with a skin blemish 122 shown thereon. The client device 104 acquires one or more images of the skin blemish 122 and sends the image(s) to the skin condition analysis server 102 via network 124. The skin condition analysis server 102 implements a neural network trained with supervised learning techniques. The skin condition analysis server 102 may identify a likely skin condition based on the uploaded images from client device 104. The skin condition analysis server 102 may retrieve one or more possible treatments for the identified likely skin condition. The diagnosed skin condition, along with a recommended treatment, are then transmitted to client device 104 and rendered on an electronic display to provide the information to a user.

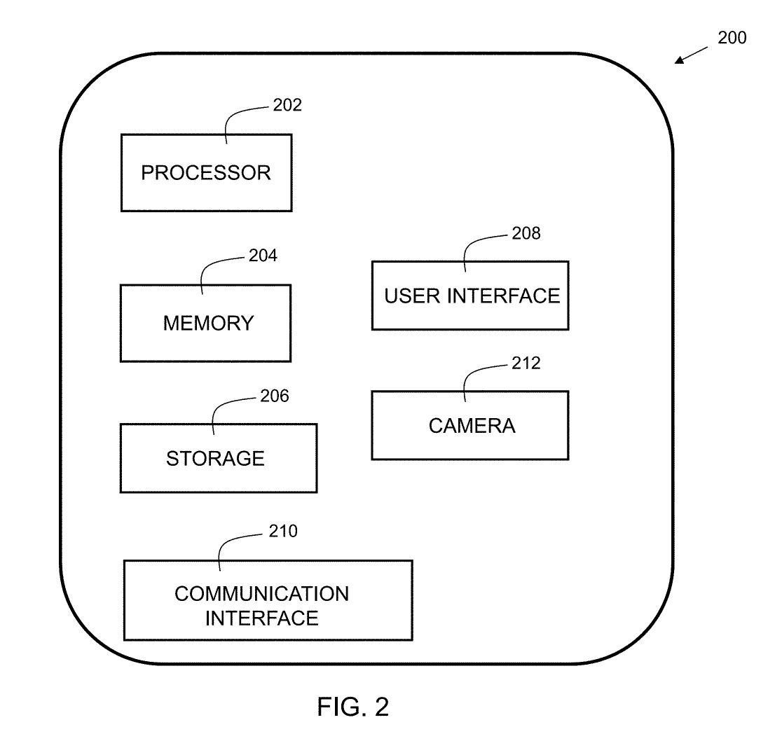

FIG. 2 is a block diagram of a client device 200 in accordance with embodiments of the present invention. In embodiments, client device 200 is an electronic device that may include a desktop computer, laptop computer, tablet computer, smartphone, and/or other suitable client device. Client device 200 may be similar to client device 104 as shown in FIG. 1. Client device 200 includes a processor 202, a memory 204 coupled to the processor 202, and storage 206. The memory 204 may be a non-transitory computer readable medium. Memory 204 may include RAM, ROM, flash, EEPROM, or other suitable storage technology. The memory 204 contains instructions, that when executed by processor 202, enable communication to/from skin condition analysis server 102 of FIG. 1. Client device 200 further includes a network communication interface 210 for performing this communication. In embodiments, network communication interface 210 includes a wireless communications interface such as a cellular data interface and/or a Wi-Fi interface. In embodiments, the storage 206 includes flash, SRAM, one or more hard disk drives (HDDs) and/or solid state disk drives (SDDs).

Device 200 may further include a user interface 208. User interface 208 may include a keyboard, monitor, mouse, and/or touchscreen, and provides a user with the ability to enter information as necessary to utilize embodiments of the present invention. In embodiments, a user uses the device 200 to access a trained neural network within the skin condition analysis server 102. Device 200 further includes a camera 212. The camera 212 is used to acquire digital images of a skin condition for uploading to skin condition analysis server 102.

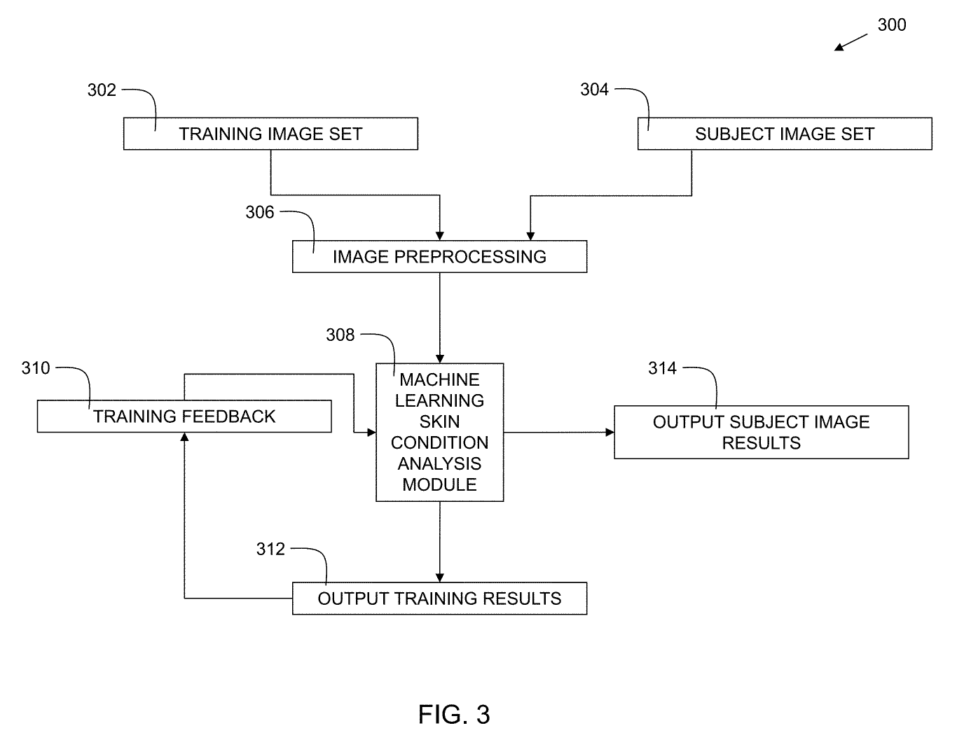

FIG. 3 is a flowchart 300 indicating process steps for embodiments of the present invention. At 302, a training image set is obtained. This can include multiple digital images in a variety of formats, including, but not limited to, JPEG, PNG, bitmap, TIFF, Targa, or another suitable format. The images can include a variety of skin conditions, including, but not limited to, herpes, acne, melanomas, and poison ivy. The images may then be preprocessed at process 306. The preprocessing can include, but is not limited to, resizing, contrast adjustment, noise removal, edge detection, gradient detection, color adjustments, and/or other suitable image processing techniques. At 308, the processed training images are then used to train machine learning skin condition analysis module. At process step 312, results of the training images are output. At process step 310, the loss (difference between computed output of training results and known output of training results) is evaluated. This loss is input back into the machine learning skin condition analysis module 308 to further refine the weights associated with neurons within a neural network of a machine learning skin condition analysis module. This process continues until the neural network of the machine learning skin condition analysis module is sufficiently trained.

Once the machine learning system is trained to a level of satisfactory performance, a subject image set 304 then undergoes image preprocessing at 306. The preprocessed subject image set is then provided to the machine learning skin condition analysis module at 308 that is already trained. Thus, the subject image set 304 may not go through the training process. Rather, the subject image diagnosis and/or treatment results are output at 314 based on results from the trained machine learning skin condition analysis module 308. In this way, a user of disclosed embodiments can diagnose skin conditions such as various types of rashes and/or other skin diseases from the convenience of their own home.

With skin conditions, many conditions may appear quite similar, so a traditional machine learning approach with neural networks may not be effective. Neural networks utilize a variety of “hyperparameters.” The structure of a neural network involves numerous hyperparameters that are used in its design, including the size and nonlinearity of each layer. The values of the hyperparameters can have a strong effect on model performance. Thus, effective skin diagnosis is a challenging task for machine learning systems.

Disclosed embodiments improve the training process of a neural network system adapted for skin condition diagnosis. Disclosed embodiments employ a second neural network within the machine learning skin condition analysis module. The second neural network receives loss data for the evaluation neural network that is used to perform the evaluation of the skin condition. Based on this information, the second neural network generates updated hyperparameters for the evaluation neural network. In this way, an improved level of accuracy for the evaluation neural network on subject images may be achieved.

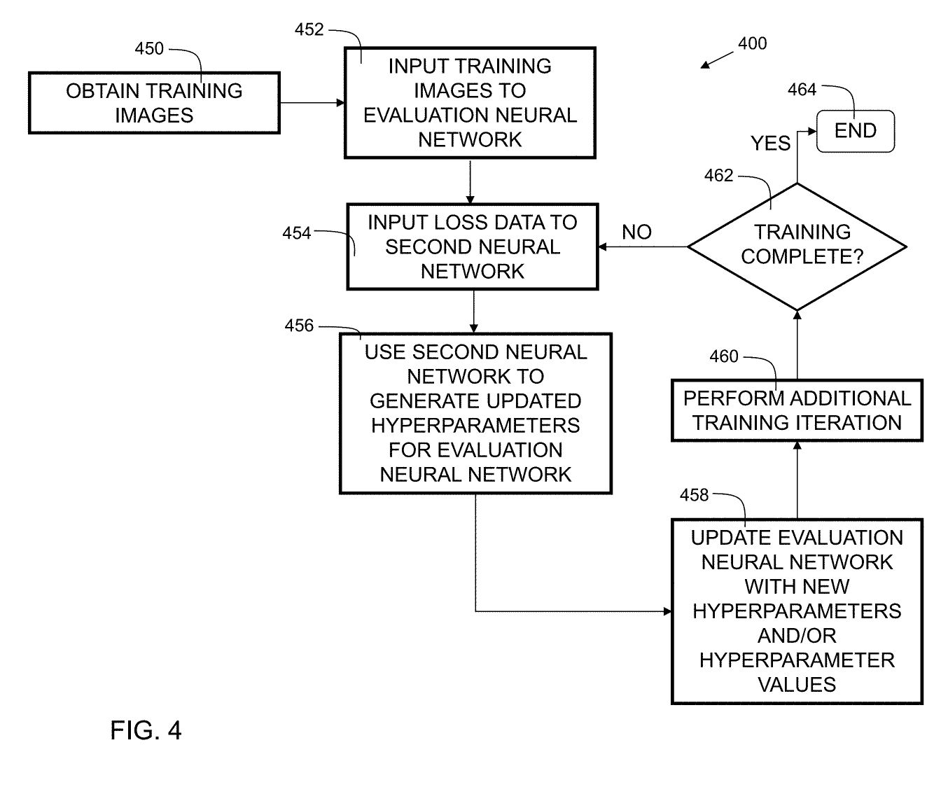

FIG. 4 is a flowchart 400 indicating additional process steps for embodiments of the present invention. At process step 450, a plurality of training images is obtained. At process step 452, the training images are input to the evaluation neural network. At process step 454, loss data from the evaluation neural network is input to a second neural network. At process step 456, the second neural network is used to generate updated hyperparameters for the evaluation neural network. The updated hyperparameters can include, but are not limited to, a training iteration parameter, a learning rate, a number of hidden layers, a configuration for one or more pooling layers, a configuration for one or more dropout layers, a size of a max pooling layer, and/or a size of an average pooling layer. Other hyperparameters may be adjusted in accordance with embodiments of the present invention. Thus, in embodiments, applying the one or more updated hyperparameters includes providing an updated training iteration parameter, providing an updated learning rate, and/or providing an updated number of hidden layers. In embodiments, providing an updated number of hidden layers comprises providing an updated number of convolutional layers. In embodiments, providing an updated number of hidden layers comprises providing an updated number of fully connected layers. In embodiments, applying the one or more updated hyperparameters includes providing an updated configuration for one or more pooling layers. In embodiments, applying the one or more updated hyperparameters includes providing an updated configuration for one or more dropout layers. In embodiments, the updated configuring of one or more pooling layers includes updating a size of a max pooling layer. In some embodiments, the updated configuring of one or more pooling layers includes updating a size of an average pooling layer. Other hyperparameters may be updated in some embodiments. Next, the evaluation neural network is updated with new hyperparameters and/or hyperparameter values at process step 458.

In process step 460, an additional training iteration is performed on the evaluation neural network using the updated hyperparameters. The process continues until the training complete criteria is met at process step 462, where a check is made to see if training is complete. The criteria for complete training can include, a number of training iterations, a convergence of weights, hyperparameters, or other parameters, and/or other suitable criteria. If the criteria are met, then the process ends at step 464. If the criteria are not met, the process continues to process step 454 to perform an additional training cycle. Thus, disclosed embodiments utilize a second neural network to receive the loss data (training data error) from a first neural network. The second neural network then generates updated hyperparameters that are used in subsequent training iterations of the first neural network. In embodiments, the first neural network is an evaluation neural network, and the second neural network is a deep neural network, and may be referred to as an optimizing neural network, since it serves the function of optimizing performance of the evaluation neural network 602.

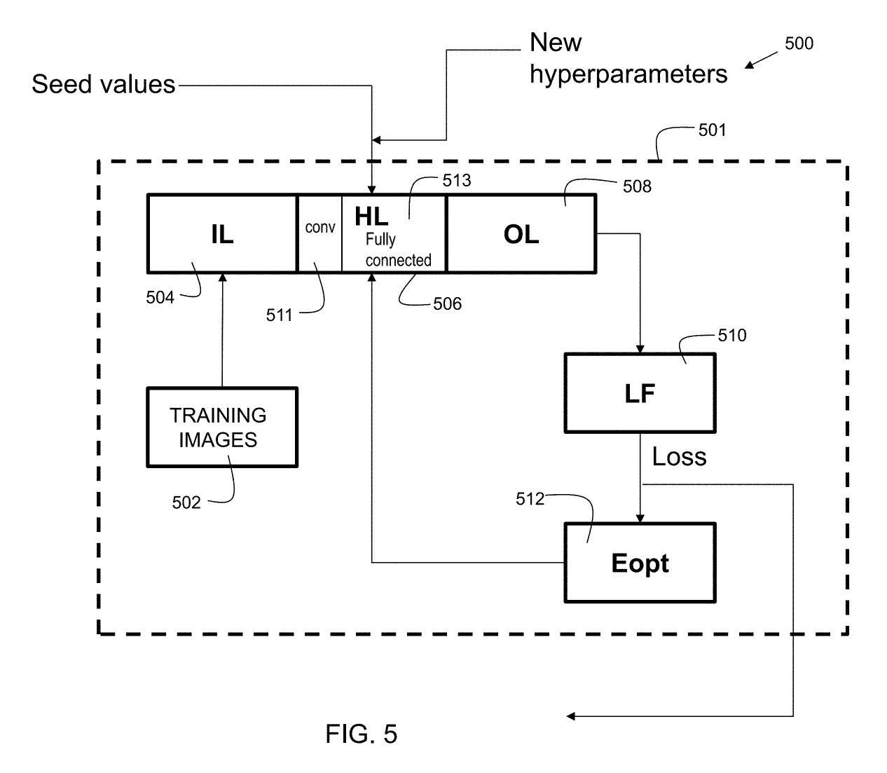

FIG. 5 is a block diagram 500 of an evaluation neural network 501 in accordance with embodiments of the present invention. Training images 502 are input to an input layer 504. One or more hidden layers 506 are configured to receive input from input layer 504, and provide an output to output layer 508. In embodiments, the hidden layers 506 can include one or more convolutional layers 511, and a plurality of fully connected layers 513. Hyperparameters can pertain to the number of convolutional layers and fully connected layers. In embodiments, these hyperparameters can be adjusted by a second neural network.

In some embodiments, the hidden layers can include convolutional layers (as determined by outputted hyperparameters) and a number of linear layers (determined by separate hyperparameters). The linear layers may each include one or more neurons. Each neuron may have an activation function. In embodiments, the activation function is a ReLU (Rectified Linear Unit) activation function. In other embodiments, another activation function may be used, including, but not limited to, sigmoid, and/or tanh functions. In yet other embodiments, the activation function is an ELU (exponential linear unit).

The output of the output layer 508 is input to a loss function analyzer 510 that outputs a loss value based on the output of output layer 508. The loss is input to an evaluation optimizer 512. The evaluation optimizer implements a function that takes loss and adjusts the weights of elements in the evaluation neural network to minimize that loss. To start the training process, various weights and/or hyperparameters of the evaluation neural network are initialized with seed values. These seed values may be predefined or randomly generated. These seed values are used to initialize the neural network 501. Based on the output of a second neural network, new hyperparameters are input to the evaluation neural network 501 on subsequent training iterations.

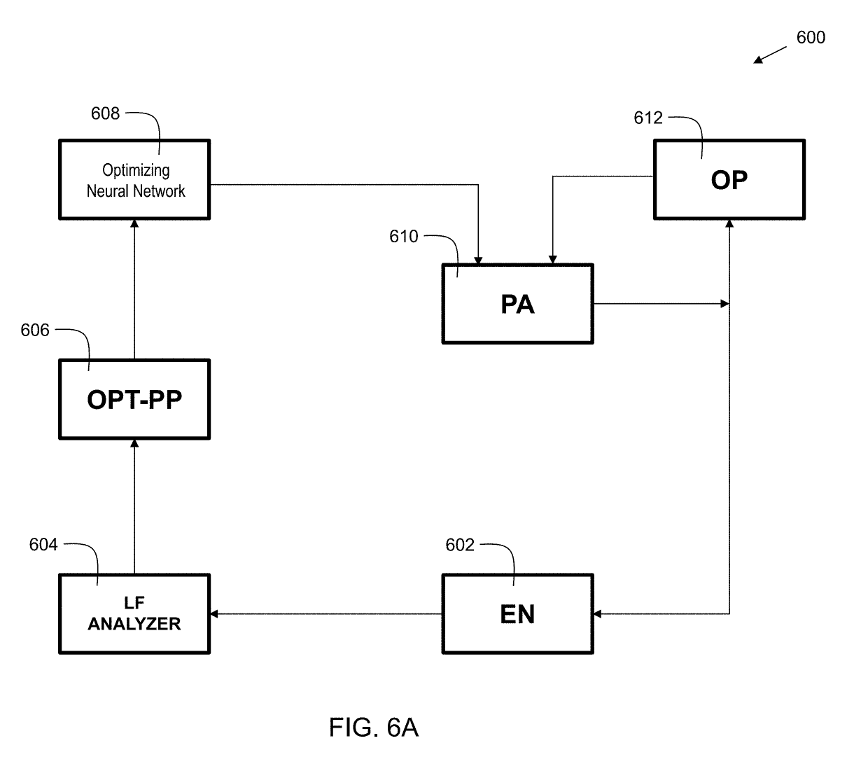

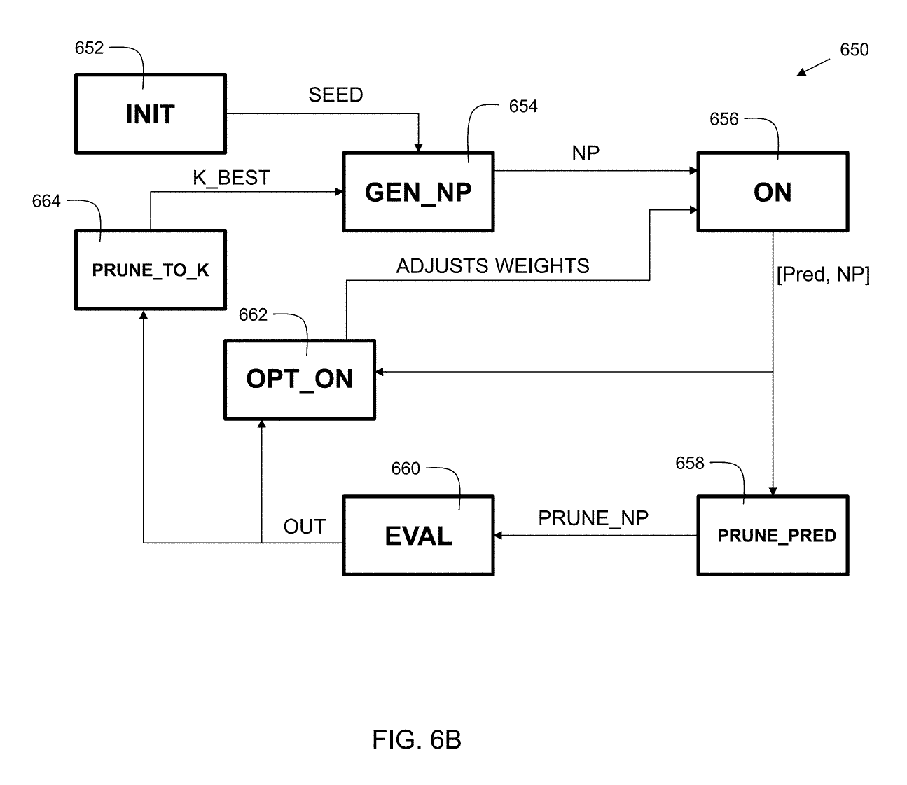

FIG. 6A is a block diagram of a feedback network 600 in accordance with embodiments of the present invention. Feedback network 600 includes an evaluation neural network 602. The evaluation neural network 602 may be similar to evaluation neural network 501 shown in FIG. 5. The output of the evaluation neural network 602 is output to a loss function analyzer 604. The loss function analyzer 604 is configured and disposed to evaluate the loss function (cost function) of the evaluation neural network 602. The output of the loss function analyzer 604 is provided to an optimizing preprocessor 606. The loss function analyzer 604 may implement a loss function (cost function) utilizing a technique such as mean squared error, Binary Cross-Entropy, Hellinger distance, Kullback-Leibler divergence, or other suitable technique. The optimizing preprocessor 606 is configured and disposed to adjust weights based on the loss function analyzer input. The output of the optimizing preprocessor 606 is provided to a second neural network 608, referred to as a deep neural network. The second neural network is configured and disposed to compute the partial derivative of the cost function with respect to each hyperparameter of the evaluation neural network 602. The output of the second neural network 608 is provided to a parameter adjustment module 610. The parameter adjustment module 610 is configured and disposed to provide one or more updated hyperparameter values to the evaluation neural network based on the partial derivatives and a previous set of hyperparameter values 612. The updated hyperparameters are then applied to the evaluation neural network 602 and additional training iterations can then be performed to further refine the evaluation neural network 602.

In some embodiments, the training process starts with generation of a random hyperparameter set. The evaluation neural network 602 is run through a training image set using these initial hyperparameters, and the resulting loss is input to the loss function analyzer 604. Then, new hyperparameters are generated using a local K search algorithm (algorithm for k-means clustering). A training image set is run on the evaluation neural network 602 using the new hyperparameters. Concurrently, the second neural network 608 makes predictions prior to each evaluation. The second network is trained with a loss function that compares the actual loss of the evaluation neural network 602 during the training run with the predicted loss from the second neural network 608. For each of the K next hyperparameters, K\hat{ }N additional hyperparameters are generated. In some embodiments, N=3. The second neural network 608 predicts the expected loss for each of the new hyperparameters. All the new hyperparameter sets from the K\hat{ }N hyperparameters are run through the second neural network 608, which selects the best K\hat{ }2 hyperparameters to use in the evaluation neural network 608. The evaluation neural network 608 is run using the K\hat{ }2 hyperparameters, again training the second neural network 608 during this phase. From this process, the best K hyperparameters are saved and used in the evaluation neural network 602. Thus, embodiments include generating K\hat{ }N hyperparameters, then filtering those to a subset of K\hat{ } hyperparameters, and then filtering those to a smaller subset of K parameters. This process can be repeated until the desired performance from the evaluation neural network is achieved.

FIG. 6B is a flow diagram of a feedback network 650 in accordance with additional embodiments of the present invention. At 652 an initialization function provides a seed. In embodiments, the seed can be an initial randomly generated or predefined hyperparameter set. At 654, the GEN_NP block generates new parameters. This can include K\hat{ }N set of hyperparameters that are generated. These hyperparameters are input to an optimization network for pruning, prior to using any of these hyperparameters for evaluation purposes. Process 654 may utilize a k-local search algorithm that randomly generates K\hat{ }N new hyperparameter sets using the current K best hyperparameters as seed values (causing the new hyperparameters sets to be randomly clustered around the K best sets with some standard deviation which can be defined at compile and/or runtime).

From 654, new hyperparameters (NP) are input to optimization neural network (ON) 656. This process accepts as input the NP and makes a prediction on each of the NP loss values when evaluated. It then outputs these parameters and the predicted loss values. This is important as the cost of making a prediction is a feed forward process and this runs multiple orders of magnitude faster than actual evaluation of each of the hyper parameters sets, which requires back-propagation. In embodiments, the aforementioned process does not use back-propagation. Thus, embodiments improve the technical field of machine learning by reducing the amount of time, power, and/or computing resources required to perform such computations.

The output of the optimization neural network 656 includes tuples of information [Pred, NP] that include the new hyperparameters (NP) and corresponding predicted loss (Pred) for those hyperparameters. At 658, the prune_pred process filters out some of the hyperparameters received from optimization network 656, and passes on some of the hyperparameters (prune_np) to the evaluation neural network 660. In embodiments, the prune_pred process 658 outputs the best K\hat{ } hyperparameters from the K\hat{ }N hyperparameters that are passed in to 658. Thus, prune_np represents a subset of hyperparameters that are output from optimization neural network 656. The output of the evaluation neural network 660 is fed to an optimization net optimizer (OPT_ON) 662, which adjusts the weights of the optimization net based on how close the predicted results were to the actual results of training images. The output of evaluation neural network 660 is also provided to prune_to_k process 664. This process receives as input the K\hat{ }2 hyperparameters that constitute the prune_np subset, and further culls them by selecting the K best hyperparameters out of the K\hat{ }2 hyperparameters that were input. The K_BEST hyperparameters are thus a second subset of the originally generated hyperparameters NP. Thus, in embodiments, the hyperparameters used in the evaluation network are obtained using the following process steps. First initial hyperparameters are established at 652. The initial hyperparameters are pruned to form a subset. A k-local search algorithm is utilized to generate K\hat{ }N new hyperparameter sets. The loss of the new hyperparameter sets is predicted. Thus, disclosed embodiments include cost predicting combined with a cluster search as an optimizing method for the trained evaluation neural network. Embodiments can include a computer-implemented method for training a neural network, comprising: obtaining a plurality of training images; providing the plurality of training images to an evaluation neural network, wherein the evaluation neural network comprises an input layer, an output layer, and one or more hidden layers configured between the input layer and the output layer; generating a first set of new hyperparameters for the evaluation neural network; pruning the plurality of the first set hyperparameters to form a subset of the first set of hyperparameters; performing a k-local search to generate a second set of new hyperparameters using the subset of the first set of hyperparameters as seeds; and evaluating the plurality of training images using the second set of new hyperparameters.

The elements shown in FIG. 6A and/or FIG. 6B may be implemented in dedicated hardware, general purpose hardware, software, or a combination of both hardware and software. Software may include implementation of routines in a high-level language such as C, C++, Python, or Perl, as well as lower level routines written in assembly language for their respective target platforms.

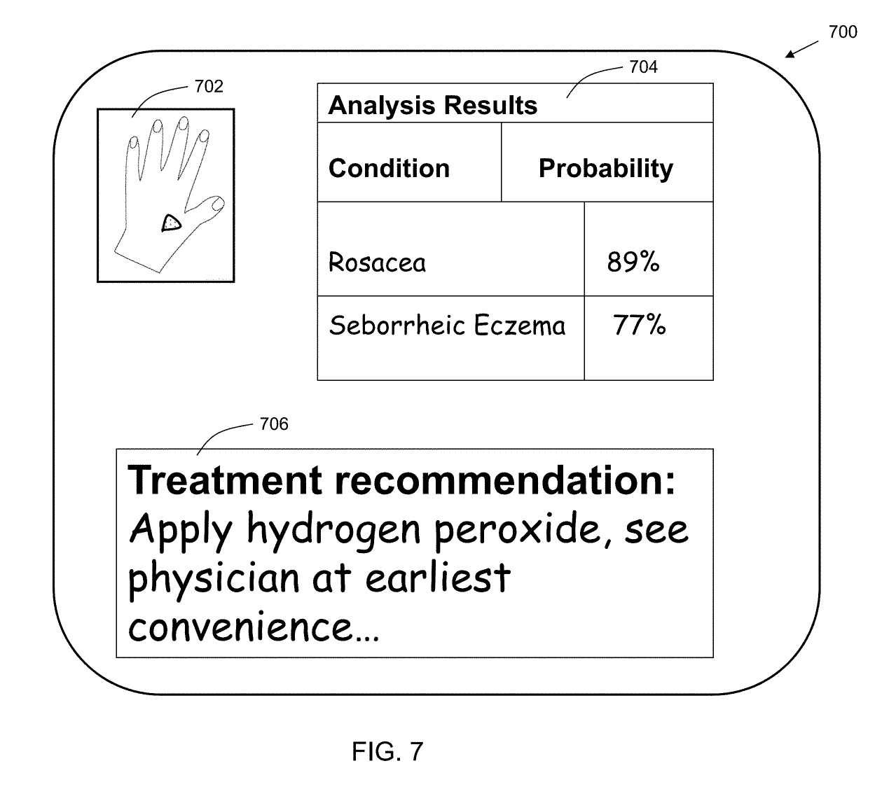

FIG. 7 is an exemplary user interface 700 showing results in accordance with embodiments of the present invention. In embodiments, this user interface is rendered on client device 104 of FIG. 1. Field 702 displays an image that is acquired by a client device (e.g. 104 of FIG. 1) and submitted for analysis. The image is provided to the skin condition analysis server 102 via network 124. The skin condition analysis server, using a machine learning system comprising a first neural network for evaluation and a second neural network configured and disposed to adjust the hyperparameters of the first neural network, provides one or more likely skin conditions, and may further provide a probability rating for each condition. The probability rating can serve as a score, confidence level, or other indicator of the likelihood of the diagnosed skin condition, based on the submitted image(s). The analysis results are provided to the client and rendered on the user interface 700 in field 704. As can be seen in field 704, two conditions (rosacea, seborrheic eczema), and two corresponding probabilities (89%, 77%) are shown. In practice, more or fewer conditions may be shown. User interface 700 further includes a treatment field 706. The treatment field 706 may show text, video, images, and/or other multimedia elements to provide treatment instructions and/or procedures for the skin condition(s) shown in field 704.

In embodiments, the information provided in the treatment field 706 is retrieved from treatment database 136. In some embodiments, the client device 104 may directly retrieve the treatment information from treatment database 136 based on a diagnosis received from skin condition analysis server 102. In some embodiments, the skin condition analysis server 102 provides an alphanumeric code corresponding to a skin condition to client device 104. The client device 104 uses the alphanumeric code as an argument in an API call to retrieve treatment information from treatment database 136.

Communication between the client device 104, skin condition analysis server 102, and/or treatment database 136 may utilize one or more protocols, including, but not limited to, HTTP, XML, and/or JavaScript Object Notation. Embodiments of the present invention may utilize a JavaScript Object Notation (JSON) web service to make a JSON call to the skin condition analysis server 102. In some examples, the JSON call is made using XML HTTP, which implements an XML HTTP object that has functionality enabling the exchange of Extensible Markup Language (XML) data directly over the Internet using the Hypertext Transfer Protocol (HTTP). The XML HTTP object allows access of the audio archiving sever data from a server, parsing the data using an XML Document Object Model (DOM), and posting XML data through a standard firewall directly to an HTTP server.

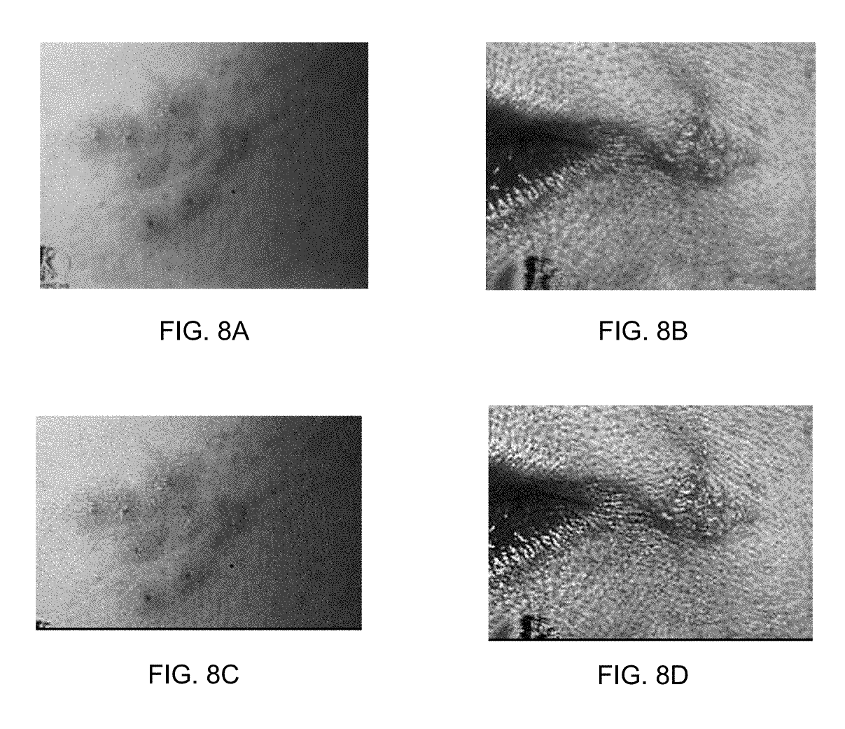

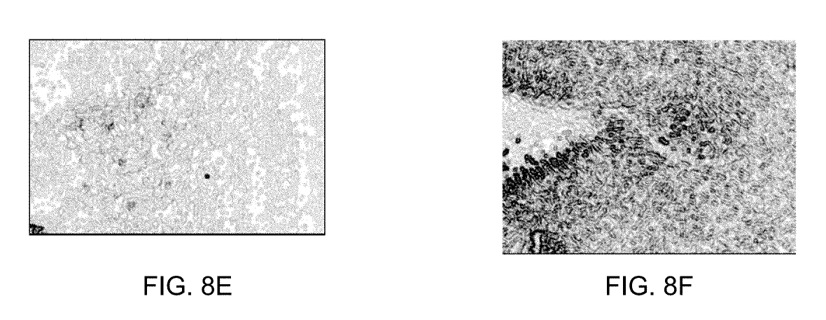

FIG. 8A-FIG. 8F show examples of training images in accordance with embodiments of the present invention. FIG. 8A shows an image of a first skin condition, and FIG. 8B shows an image of a second skin condition. These images are representative of images that may be used for training. In some embodiments, various preprocessing techniques may be applied to the images before they are submitted to the evaluation neural network for training. FIG. 8C and FIG. 8D show examples of edge enhancement preprocessing utilizing a 5×5 convolution mask. FIG. 8C is an edge enhanced image of FIG. 8A, and FIG. 8D is an edge enhanced image of FIG. 8B. FIG. 8E is an edge detection image of FIG. 8A utilizing a Sobel Feldman edge detection process. FIG. 8F is an edge detection image of FIG. 8B utilizing a Sobel Feldman edge detection process. In other embodiments, a Canny edge detection process may be used on the training images. These are merely examples of image preprocessing techniques that may be used with embodiments of the present invention. Other preprocessing techniques may be used in other embodiments of the present invention.

As can now be appreciated, disclosed embodiments improve the technical field of machine learning and further provide a skin condition analysis system. A user, using a client device such as a smartphone, takes one or more images of a skin condition. The images are uploaded to a skin condition analysis server that implements an evaluation neural network that is trained to analyze skin conditions. The evaluation neural network has hyperparameters that are adjusted utilizing a second neural network to achieve a higher level of diagnostic effectiveness. The client device then retrieves the diagnosis, and renders the diagnosis, along with a corresponding treatment regiment, on a user interface of the client device. In this way, access to effective skin condition treatment is greatly increased. This can serve to provide quicker remedies, thereby reducing overall healthcare costs.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, circuits, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

What is claimed is:

1. A computer-implemented method for training a neural network, comprising:
obtaining a plurality of training images;
providing the plurality of training images to an evaluation neural network, wherein the evaluation neural network comprises an input layer, an output layer, and one or more hidden layers configured between the input layer and the output layer;
providing an output of the evaluation neural network into a loss function analyzer, wherein the loss function analyzer is configured and disposed to evaluate a loss function of the evaluation neural network;
providing an output of the loss function analyzer to an optimizing preprocessor, wherein the optimizing preprocessor is configured and disposed to adjust weights based on the loss function analyzer input;
providing an output of the optimizing preprocessor to a second neural network, wherein the second neural network is configured and disposed to compute partial derivatives of one or more hyperparameters of the evaluation neural network;
providing an output of the second neural network to a parameter adjustment module, wherein the parameter adjustment module is configured and disposed to provide one or more updated hyperparameter values to the evaluation neural network based on the partial derivatives and a previous set of hyperparameter values;
applying the one or more updated hyperparameters to the evaluation neural network; and
training the evaluation neural network, using the plurality of training images.

2. The method of claim 1, wherein applying the one or more updated hyperparameters includes providing an updated training iteration parameter.

3. The method of claim 1, wherein applying the one or more updated hyperparameters includes providing an updated learning rate.

4. The method of claim 1, wherein applying the one or more updated hyperparameters includes providing an updated number of hidden layers.

5. The method of claim 4, wherein providing an updated number of hidden layers comprises providing an updated number of convolutional layers.

6. The method of claim 4, wherein providing an updated number of hidden layers comprises providing an updated number of fully connected layers.

7. The method of claim 1, wherein applying the one or more updated hyperparameters includes providing an updated configuration for one or more pooling layers.

8. The method of claim 1, wherein applying the one or more updated hyperparameters includes providing an updated configuration for one or more dropout layers.

9. The method of claim 7, wherein the updated configuring of one or more pooling layers includes updating a size of a max pooling layer.

10. The method of claim 1, wherein the parameter adjustment module is configured and disposed to perform the steps of:
generating a first set of new hyperparameters for the evaluation neural network;
pruning the plurality of the first set hyperparameters to form a subset of the first set of hyperparameters; and
performing a k-local search to generate a second set of new hyperparameters using the subset of the first set of hyperparameters as seeds.

11. The method of claim 1, further comprising:
inputting a subject image to the evaluation neural network;
obtaining a skin condition diagnosis from the evaluation neural network;
obtaining a treatment procedure corresponding to the skin condition diagnosis; and
rendering the skin condition diagnosis and the treatment procedure on a user interface of an electronic device.

12. The method of claim 1, wherein the plurality of training images comprises a variety of skin conditions.

13. The method of claim 1, wherein the variety of skin conditions comprises one or more of herpes, acne, melanomas, and poison ivy.

14. An electronic computation device, comprising:
a processor;
a memory coupled to the processor, the memory containing instructions, that when executed by the processor, perform the steps of:
obtaining a plurality of training images;
providing the plurality of training images to an evaluation neural network, wherein the evaluation neural network comprises an input layer, an output layer, and one or more hidden layers configured between the input layer and the output layer;
providing an output of the evaluation neural network into a loss function analyzer, wherein the loss function analyzer is configured and disposed to evaluate a loss function of the evaluation neural network;
providing an output of the loss function analyzer to an optimizing preprocessor, wherein the optimizing preprocessor is configured and disposed to adjust weights based on the loss function analyzer input;
providing an output of the optimizing preprocessor to a second neural network, wherein the second neural network is configured and disposed to compute partial derivatives of one or more hyperparameters of the evaluation neural network;
providing an output of the second neural network to a parameter adjustment module, wherein the parameter adjustment module is configured and disposed to provide one or more updated hyperparameter values to the evaluation neural network based on the partial derivatives and a previous set of hyperparameter values;
applying the one or more updated hyperparameters to the evaluation neural network; and
training the evaluation neural network, using the plurality of training images.

15. The electronic computation device of claim 14, wherein the memory further comprises instructions, that when executed by the processor, perform the step of updating the one or more hidden layers by providing an updated number of convolutional layers.

16. The electronic computation device of claim 14, wherein the memory further comprises instructions, that when executed by the processor, performs the step of updating the one or more hidden layers by:
providing an updated number of fully connected layers.

17. The electronic computation device of claim 14, wherein the memory further comprises instructions, that when executed by the processor, performs the step of providing an updated configuration for one or more pooling layers.

18. A computer program product for an electronic computation device comprising a computer readable storage medium having program instructions embodied therewith, the program instructions executable by a processor to cause the electronic computation device to perform the steps of:
obtaining a plurality of training images;
providing the plurality of training images to an evaluation neural network, wherein the evaluation neural network comprises an input layer, an output layer, and one or more hidden layers configured between the input layer and the output layer;
providing an output of the evaluation neural network into a loss function analyzer, wherein the loss function analyzer is configured and disposed to evaluate a loss function of the evaluation neural network;
providing an output of the loss function analyzer to an optimizing preprocessor, wherein the optimizing preprocessor is configured and disposed to adjust weights based on the loss function analyzer input;
providing an output of the optimizing preprocessor to a second neural network, wherein the second neural network is configured and disposed to compute partial derivatives of one or more hyperparameters of the evaluation neural network;
providing an output of the second neural network to a parameter adjustment module, wherein the parameter adjustment module is configured and disposed to provide one or more updated hyperparameter values to the evaluation neural network based on the partial derivatives and a previous set of hyperparameter values;
applying the one or more updated hyperparameters to the evaluation neural network; and
training the evaluation neural network, using the plurality of training images.

19. The computer program product of claim 18, wherein the computer readable storage medium includes program instructions executable by the processor to cause the electronic communication device to perform the step of updating the one or more hidden layers by providing an updated number of convolutional layers.

20. The computer program product of claim 18, wherein the computer readable storage medium includes program instructions executable by the processor to cause the electronic communication device to perform the step of updating the one or more hidden layers by providing an updated number of fully connected layers.
